(19) United States
(12) Patent Application Publication
(10) Pub. No.: US 2023/0381459 A1
(43) Pub. Date: Nov. 30, 2023

(54) INTRAVASCULAR CATHETER WITH INTEGRATED GUIDE STRUCTURE

(71) Applicant: VENOCARE, INC., Doral, FL (US)

(72) Inventors: Amir BELSON, Savyon (IL); Raul LEYTE-VIDAL, Doral, FL (US)

(21) Appl. No.: 18/248,210

(22) PCT Filed: Oct. 7, 2021
(86) PCT No.: PCT/US2021/054046

§ 371 Date: Apr. 6, 2023

Related U.S. Application Data
(60) Provisional application No. 63/088,979, filed on Oct. 7, 2020.

(51) Int. Cl. A61M 25/06; A61M 25/00

(52) U.S. Cl. CPC A61M 25/0606; A61M 2025/0687; A61M 25/0041; A61M 25/0631

(57) ABSTRACT

Intravascular access is achieved by introducing a catheter having a guide element recess placed to receive the contoured distal end of a guide element. The guide element may be advanced along an outer surface of an access needle to form a loop beyond the distal end of the needle. The deployed guide element may form a partial or complete loop once beyond the distal end of the needle. After the catheter has been properly positioned, the access needle and guide element may be removed by automatic withdrawal leaving the catheter in place within a blood vessel and available for use.

### CROSS REFERENCE TO RELATED APPLICATIONS

This application claims the benefit of U.S. Provisional Patent Application Ser. No. 63/088,979 entitled “INTRAVASCULAR CATHETER WITH INTEGRATED GUIDE STRUCTURE,” filed on Oct. 7, 2020.

### INCORPORATION BY REFERENCE

All publications and patent applications mentioned in this specification are herein incorporated by reference to the same extent as if each individual publication or patent application was specifically and individually indicated to be incorporated by reference.

### STATEMENT AS TO FEDERALLY SPONSORED RESEARCH

This invention was not made with Government support.

### FIELD

The present invention relates generally to methods and systems for performing vascular access. More particularly, the present invention provides a catheter and needle assembly with an integrated guide element or structure for transcutaneous insertion of the catheter into a patient's arterial or venous vasculature.

### BACKGROUND

One common type of vascular access is called venipuncture. Venipuncture refers generally to the process of obtaining intravenous access for any one of a variety of purposes, including intravenous infusion, therapy, blood sampling, and the like. In the hospital, for example, venipuncture is commonly used to place a small intravenous catheter for delivering intravenous fluids, drug delivery, blood sampling and the like.

While venipuncture and other forms of vascular access in relatively healthy patients can be a simple matter, such access is often needed in patients who are not healthy and may have small, tortuous, collapsed, fragile, and/or difficult to locate arteries and/or veins. In such patients, venipuncture and other forms of vascular access can be very challenging, particularly to less experienced phlebotomists, paramedics, nurses, and other health care practitioners.

In addition to difficult access, many vascular catheter placement systems can result in accidental punctures and/or accidental needle contamination during or after placement of the intravascular catheter. Still further, some conventional catheter placement devices employ relatively complex deployment handle movements that lead to increases both cost and complexity. Additionally, conventional handle placement and movements can obscure the presence and status of the needle and guide structure or guide element components of the tool, thus making use of the insertion tool less intuitive.

For these reasons, it would be desirable to provide improved methods, systems, and tools for deploying intravascular catheters using needles and guide structures. It would be particularly desirable to provide simplified deployment systems and assemblies having fewer components and, even more desirably, to provide components which are clearly visible to the user and configured to be utilized and manipulated in a straightforward, intuitive manner. At least some of these objectives will be met by the various embodiments that follow.

### SUMMARY OF THE DISCLOSURE

In general, in one embodiment, an intravascular catheter assembly includes a handle having a proximal end and a distal end, a slot in a surface of the handling having a distal end and a proximal end, an actuation button on the handle coupled to a needle carrier, a tubular catheter body having a distal end, a guide element cutout in the distal end, a proximal end, and a lumen extending between the proximal end and the distal end, the proximal end of the catheter body coupled to the distal end of the handle, an access needle disposed in the tubular catheter body lumen having a tissue-penetrating distal tip extending distally beyond the distal end of the tubular catheter body and a proximal end coupled to the needle carrier, a guide element having a proximal end and a distal end, the guide element positioned within the catheter body lumen and adjacent to an outer wall of the access needle, a distal end portion of the guide element is shaped to provide a uniform transition when the distal end portion is within the guide element cutout, wherein the access needle and the guide element may be withdrawn together from the tubular catheter body after the distal most portion of the guide element has been advanced from the tubular catheter body lumen along an outer wall of the access needle to a position distal to the tissue-penetrating distal tip of the access needle; and a slide coupled to the proximal end of the guide element so that distal advancement of the slide advances the distal tip end portion of the guide element from a position within the guide element cutout distally along the outer wall of the access needle.

This and other embodiments can include one or more of the following features. When the slide is in a distal most position, a distal portion of the guide element can curve beyond the tissue penetrating distal tip of the access needle. When the slide is in a distal most position, a distal portion of the guide element can curve from a position above the access needle, beyond the tissue penetrating distal tip of the access needle to a position below the access needle. The angle of the curve formed by the distal portion of the guide element relative to the tissue penetrating distal tip of the access needle can be less than 360 degrees. The angle of the curve formed by the distal portion of the guide element relative to the tissue penetrating distal tip of the access needle can be more than 270 degrees. The angle of the curve formed by the distal portion of the guide element relative to the tissue penetrating distal tip of the access needle can be less than 270 degrees. The angle of the curve formed by the distal portion of the guide element relative to the tissue penetrating distal tip of the access needle can be more than 180 degrees. The slide can be disposed over a proximal region of the access needle. The intravascular catheter assembly and further include a housing attached to a proximal end of the access needle, wherein the slide can be disposed over the housing. The access needle can be fixedly secured to a distal end of the housing and the tubular catheter body can be detachably secured to the distal end of the housing. The slide can advance the guide element beyond the distal end of the catheter and wherein the housing, access needle and guide element may be detached and removed from the catheter after the catheter is in place. A proximal region of the catheter can be disposed within the housing and wherein the proximal end of the catheter and the proximal end of the access needle are configured to be engaged by the slide to advance the catheter and needle in tandem with the guide element relative to the housing after the guide element has been extended distally beyond the distal end of the catheter. The access needle can have a lumen and the guide element can be disposed outside of the access needle lumen. The housing can have an axial slot and the proximal end of the guide element can be connected to the slide allowing movement along the axial slot. The axial slot can be closed over distal and proximal regions of the access needle. A link between the guide element and the slide can be disposed in the slot with a length of travel defined by closed ends of the slot. The guide element can be slidably disposed within a guide structure or guide element within the catheter body lumen. The guide structure can maintain the guide element in a position along an upper most portion of the access needle. The guide structure can be a pair of features extending from the interior wall of the catheter body and protruding into the catheter body lumen. When the guide element or the guide structure is positioned between the pair of features extending the guide structure can be positioned along a superior aspect of an exterior wall of the access needle. The guide structure can have a distal most portion adjacent to the guide element cutout and a proximal portion more than 1 cm from the guide element cutout. The guide structure can have a distal most portion adjacent to the guide element cutout and a proximal portion wherein the guide structure or guide element is continuous along the interior wall of the catheter body lumen from the proximal end to the distal end. The tubular catheter body can have a proximal hub with a hemostasis valve, wherein the access needle slidably can extend through the hemostasis valve. The slide can have a distal face which mates with a proximal face of the needle carrier when the slide is fully advanced distally to extend the guide element. The slide can detachably lock to the proximal end of the needle carrier when the distal face mates with the proximal face. The intravascular catheter assembly can further include a first configuration when the slide is in a proximal most position wherein the distal end of the guide element is in the guide element cutout and the tissue penetrating distal tip of the needle is distal to the distal most end of the tubular catheter body, a second configuration when the slide is in a distal most position wherein the distal most end of the guide element is beyond the guide cutout and curved beyond the tissue penetrating distal tip of the needle, and a third configuration when the slide is in a position proximal to the distal most position and the needle and the guide element are at least partially retracted into the housing. In the first configuration the distal tip of the access needle can extend beyond the distal end of the tubular catheter body by a distance in a range from 0.1 mm to 20 mm, and a proximal end of the slide is retracted from the proximal end of the tubular catheter body by a distance in a range from 10 mm to 100 mm In the second configuration the length of the guide element extending beyond the guide cut out of the tubular catheter body can be from 5 mm to 100 mm. The guide element can have a length greater than a width and a width that is greater than a thickness. The guide element can be made entirely or partially of a polymeric material, a PTFE, a Nylon. The guide element can be made entirely or partially of metal, comprising Nitinol, comprising Elgiloy or similar materials. The guide element can further include an upper surface conforming to a curvature of an interior wall of the catheter body lumen and a lower surface conforming to a curvature of an outer wall of the access needle. The catheter can be configured for use as a catheter component of an infusion set, a catheter component in a blood collection set, a catheter component in a safety winged blood collection set, or a catheter component in an intravenous catheter.

In general, in one embodiment, an intravascular catheter assembly includes a tubular catheter body with a proximal hub, a guide element cut out in a distal most end and a catheter body lumen extending from a proximal end to a distal end, an access needle disposed in the catheter body lumen and having a tissue-penetrating distal tip extending distally beyond the distal end of the tubular catheter body, a guide element within the catheter body lumen and outside of the access needle, the guide element having an upper surface conforming to a curvature of an interior wall of the catheter body lumen, a lower surface conforming to a curvature of an outer wall of the access needle and distal tip shaped to form a uniform transition with the guide element cut out in the distal most end of the catheter body and a slide coupled to a proximal end of the guide element so that distal advancement of the slide advances the distal tip of the guide element from a fist position at the guide element cut out to a second position wherein a distal position of the guide element curves beyond the tissue penetrating distal tip of the access needle, wherein the slide has a distal face which mates with a proximal face of the needle carrier when the slide is fully advanced distally to extend the guide element.

This and other embodiments can include one or more of the following features. The slide can detachably lock to the proximal hub when the distal face of the slide mates with the proximal face of the needle carrier. The guide element can have a length greater than a width and a width that is greater than a thickness. The guide element can be a polymer.

In general, in one embodiment, a method of accessing a blood vessel of a patient, includes advancing a tissue penetrating distal tip of an access needle of an intravascular catheter assembly into a blood vessel of the patient, advancing a guide structure from a first position where a distal portion of the guide structure forms a uniform transition within a guide cutout in a distal portion of a catheter hub to a second position where a portion of the guide structure is advanced along an outer wall of the access needle beyond the distal most portion of the catheter hub, advancing the guide structure to a third position beyond the tissue penetrating distal tip of the access needle along the blood vessel of the patient, advancing a distal portion of the catheter hub in the blood vessel along the guide structure, and automatically withdrawing the needle and the guide structure into the intravascular catheter assembly.

This and other embodiments can include one or more of the following features. The method of accessing a blood vessel can further include ceasing to perform the step of advancing a tissue penetrating distal tip of an access needle of the intravascular catheter assembly when bleed back is observed in the intravascular catheter assembly. The method of accessing a blood vessel can further include ceasing to perform the step of advancing a tissue penetrating distal tip of an access needle when a distal most portion of a catheter hub of the intravascular catheter assembly is within the blood vessel of the patient. The method of accessing a blood vessel can further include ceasing to perform the step of advancing a tissue penetrating distal tip of an access needle when a distal most portion of a guide structure of the intravascular catheter assembly is within the blood vessel of the patient. After performing the step of advancing the guide structure beyond the tissue penetrating distal tip of the access needle a distal portion of the guide element can curve beyond the tissue penetrating distal tip of the access needle. After performing the step of advancing the guide structure beyond the tissue penetrating distal tip of the access needle a distal portion of the guide element can curve from a position above the access needle, beyond the tissue penetrating distal tip of the access needle to a position below the access needle. The angle of the curve formed by the distal portion of the guide element relative to the tissue penetrating distal tip of the access needle can be less than 360 degrees. The angle of the curve formed by the distal portion of the guide element relative to the tissue penetrating distal tip of the access needle can be more than 270 degrees. The angle of the curve formed by the distal portion of the guide element relative to the tissue penetrating distal tip of the access needle can be less than 270 degrees. The angle of the curve formed by the distal portion of the guide element relative to the tissue penetrating distal tip of the access needle can be more than 180 degrees.

In general, in one embodiment, an intravascular catheter includes a hub having a proximal end and a distal end, a tubular body extending from the hub distal end to a distal tip, the tubular body having an outer wall and an inner wall, a lumen within the hub and the tubular body bounded by the tubular body inner wall, the lumen having a proximal opening in the hub proximal end and a distal opening at the tubular body distal tip, and a guide element cutout in the tubular body distal tip.

This and other embodiments can include one or more of the following features. The intravascular catheter can further include a guide element recess formed within and extending along at least a distal portion of the tubular body inner wall. The intravascular catheter can further include a guide element recess formed within and extending along the tubular body inner wall. The intravascular catheter can further include a guide element recess formed within and extending along the tubular body inner wall from the hub distal end to the tubular body distal end. The guide element recess can be wider than the guide element cutout. The intravascular catheter can further include a guide element track along the tubular body inner wall in longitudinal alignment with the guide element cutout. The guide element track along the tubular body inner wall in longitudinal alignment with the guide element cutout can be formed by a recessed portion of the tubular body inner wall alone or in combination with one or more guide element track features extending from or formed in the tubular body lumen inner wall wherein a portion of the guide element track conforms to at least a portion of a guide element upper surface contour, a guide element lower surface contour, a guide element left side contour, or a guide element right side contour. The intravascular catheter can be configured for use as a catheter component of an infusion set, a catheter component in a blood collection set, a catheter component in a safety winged blood collection set, or a catheter component in an intravenous catheter.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a perspective view of an embodiment of an intravascular access device having a catheter, guide element and needle assembly.

FIG. 2 is a cross section view of the intravascular access device of FIG. 1 showing the catheter, guide element and needle assembly, actuation button, support rail and slider in the proximal position.

FIG. 3 is a top perspective view of the distal housing and catheter hub of FIG. 1 showing details of the needle, guide element and needle carrier within the interior of catheter hub.

FIG. 4 is an enlarged top perspective view of the distal housing and catheter hub of FIG. 3 showing details of the needle, guide element, needle carrier and needle carrier slot within the interior of the catheter hub.

FIG. 5 is a perspective view of a longitudinal cross section of the intravascular access device of FIG. 3 showing additional details of the needle carrier in relation to the actuation button and the guide element to the support rail and the access needle outer wall.

FIG. 6 is an upper view of a portion of the intravascular catheter of FIG. 1 proximal to the view of FIG. 4 showing additional details of the distal end of the needle carrier in relation to the guide element and support rail that are within the needle carrier longitudinal slot.

FIG. 7 is an upper view of a portion of an interior view similar to FIG. 6 that shows the proximal end of the needle carrier in relation to the slider, guide element and support rail.

FIG. 8 is an upper perspective view of a distal portion of the intravascular access device of FIGS. 1 and 3 showing the catheter hub interior including the needle carrier, the needle, the guide element and the support rail.

FIG. 9 is perspective proximal view of the components seen in FIG. 8.

FIG. 10 is an enlarged view of the distal end of the intravascular device of FIG. 1 showing the distal end of the access needle in relation to the distal tip of the catheter with a guide element cut out and the distal end of the guide element within the guide element cut out.

FIG. 11 is a distal end on view of a cross section of the vascular access device of FIG. 10 showing the relationship of the catheter inner wall, the guide element and the access needle outer wall.

FIG. 12 is an enlarged view of the central portion of the view of FIG. 11 showing a recessed portion of the catheter lumen wall, the upper surface of the guide element adjacent to the lumen wall recessed portion and the guide element lower surface adjacent and conforming to the access needle outer wall.

FIG. 13 is an enlarged view of the central portion of the view of FIG. 11 similar to FIG. 12 providing additional explanation of the access needle outer wall, the catheter lumen wall in relation to the various contours of the guide element positioned between these structures of the intravascular device.

FIG. 14 is a perspective view of the distal end of the catheter and access needle of FIG. 10 and FIG. 1 after the slider has been moved from a proximal position as shown in FIG. 1 to a distal position where the guide element is advanced distally along an outer wall of the access needle to form a loop in a position distal to the tip of the access needle.

FIG. 15 is an enlarged view of the guide element loop of FIG. 14.

### DETAILED DESCRIPTION

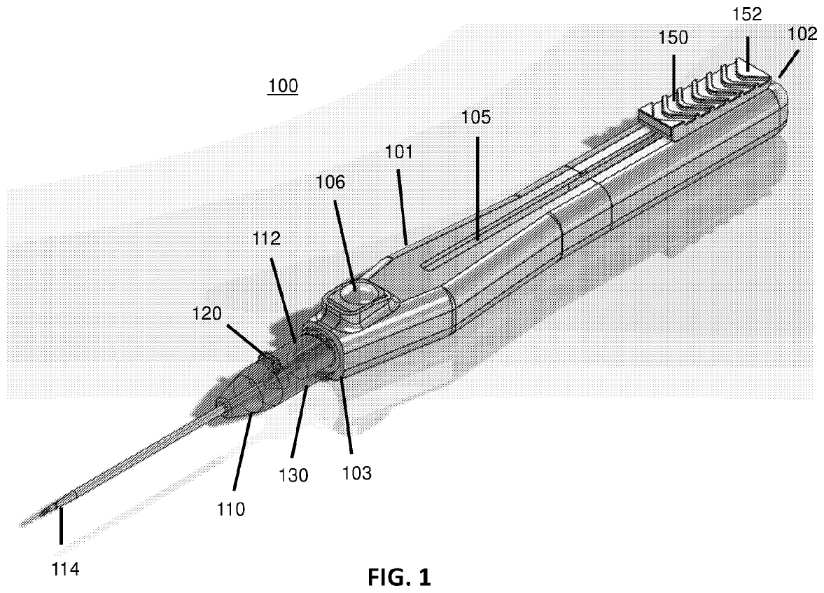

FIG. 1 is a perspective view of an embodiment of an intravascular access device 100 having a catheter 110, guide element 120 and needle assembly. In this configuration, the intravascular catheter with integrated guide structure or guide element is ready for use to position the catheter within a selected portion of the vasculature or a lumen based on clinical need. The catheter hub 110 is coupled to the distal end 103 of the handle 101 with an access needle 140 in place within the catheter lumen 116. The catheter hub 110 is transparent in this view in order to see the position of the needle carrier 130 and guide element 120.

The handle or housing 101 has a proximal end 102 and a distal end 103. A slot 105 runs along the length providing access for the slide 150 to engage with the guide element 120 and move along the support rail 109 within the interior of the handle 101. The slide 150 includes a grip 152 and is shown in the proximal most position which is used when the needle 140 is positioned in the catheter 110 and ready for insertion into the vasculature of a patient. The catheter 110 has a distal end 114 and a proximal end 112 and an interior lumen 116 along the length. The catheter proximal end 112 is adapted and configured to engage with the handle distal end 103. The catheter proximal end 112 is also configured to engage with conventional medical tubing connections once inserted and the handle 101 is removed. For example, the catheter proximal end 112 may comprise features to engage with a luer lock, fluid coupling or other suitable medical connector or fitting.

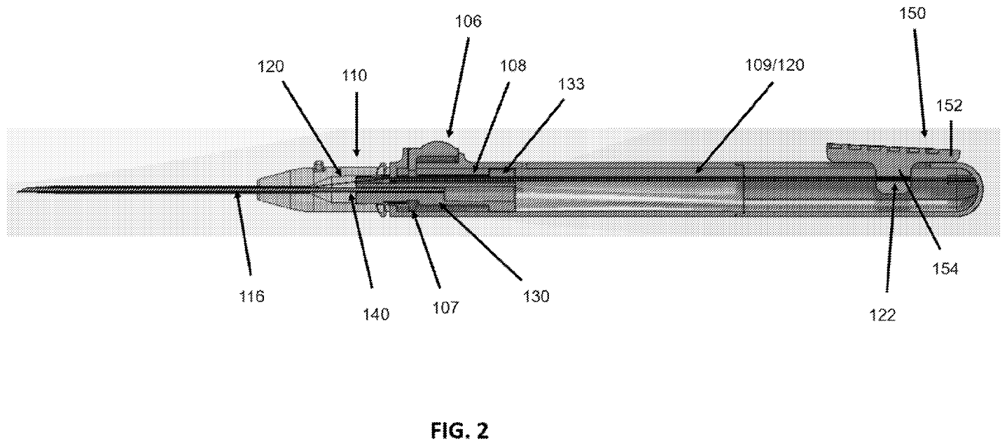

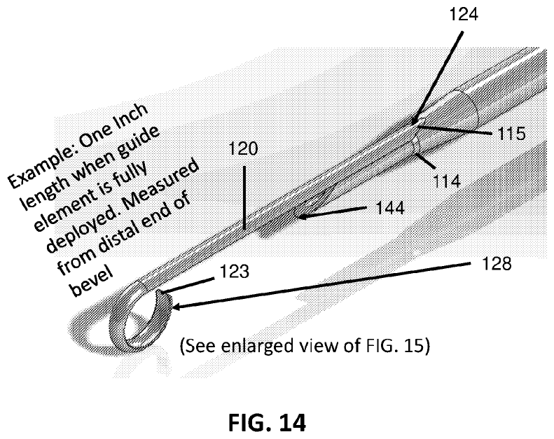

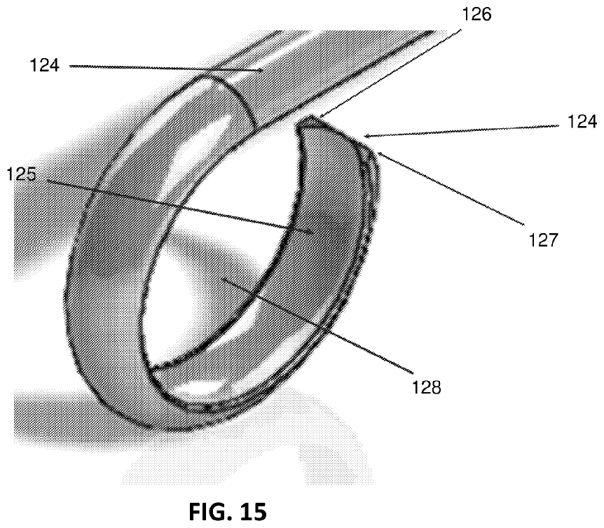

FIG. 2 is a cross section view of the intravascular access device of FIG. 1 showing the catheter 110, guide element 120 and needle carrier 130, actuation button 106, support rail 109 and slide 150 in the proximal position. This is a “ready to use” configuration. The guide element proximal end 122 is attached to the slide tab 154. The guide element 120 extends along the support rail 109 beyond the needle carrier distal end 132, into and along the catheter lumen 116. The guide 120 is between the needle outer wall 148 and the catheter lumen inner wall 115. As a result, when the slide 150 is advanced along the slot 105, the guide element is advanced along the support rail 109, along and beyond the distal end of the catheter and the needle forming an atraumatic loop or partial loop or curve to cover the needle sharp distal end 144. (see FIGS. 14 and 15).

The catheter lumen 116 is also visible in this view. The needle 140 has a sharp distal end 144 used to penetrate into the skin and vessel wall. The access needle proximal end 142 terminates within the needle carrier 130. When the activation button is released, the activation element 108 moves the needle carrier 130 towards the housing proximal end 102. The movement of the needle carrier 130 within the handle 101 is long enough so that the sharp distal end 144 of the needle 140 is completely within the housing 101. The needle carrier 130 also includes a support rail slot 138 along its length sized to receive the support rail 109.

An actuation element 108 is included but not shown in this view in order to see the details of the needle carrier 130. The actuation element 108 may be a spring such as a coil spring or a wave spring or other suitable compression element. When the actuation button 106 is depressed, the actuation button latch 107 disengages from the needle carrier distal groove 134, when then allows the actuation element 108 to expand and propel the needle carrier 130 along with needle 140 towards the handle proximal end 102.

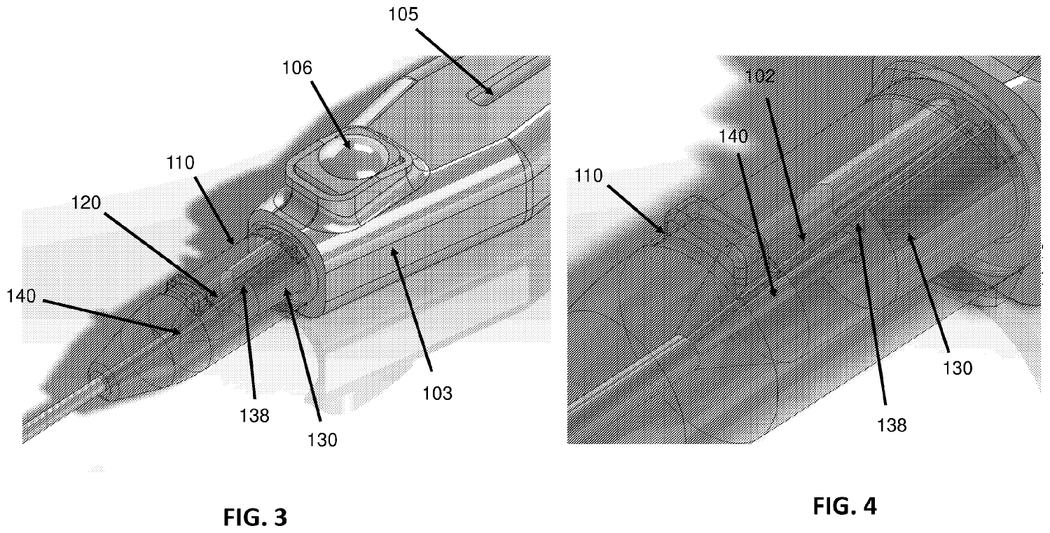

FIG. 3 is a top perspective view of the distal housing 103 and catheter hub 110 of FIG. 1 showing details of the needle 140, guide element 120 and needle carrier 130 within the interior of catheter hub 110. The guide element 120 is shown exiting the distal end of the support rail and entering the space between the needle outer wall and the inner wall of the catheter lumen.

FIG. 4 is an enlarged top perspective view of the distal housing 103 and catheter hub 110 of FIG. 3 showing details of the needle 140, guide element 120, needle carrier 130 and needle carrier support rail slot 138 within the interior of the catheter hub 110. In this view the needle 140 is shown exiting the catheter lumen 116 and entering the needle carrier lumen 136. The proximal end of needle 140 terminates within and is secured to the needle carrier 130.

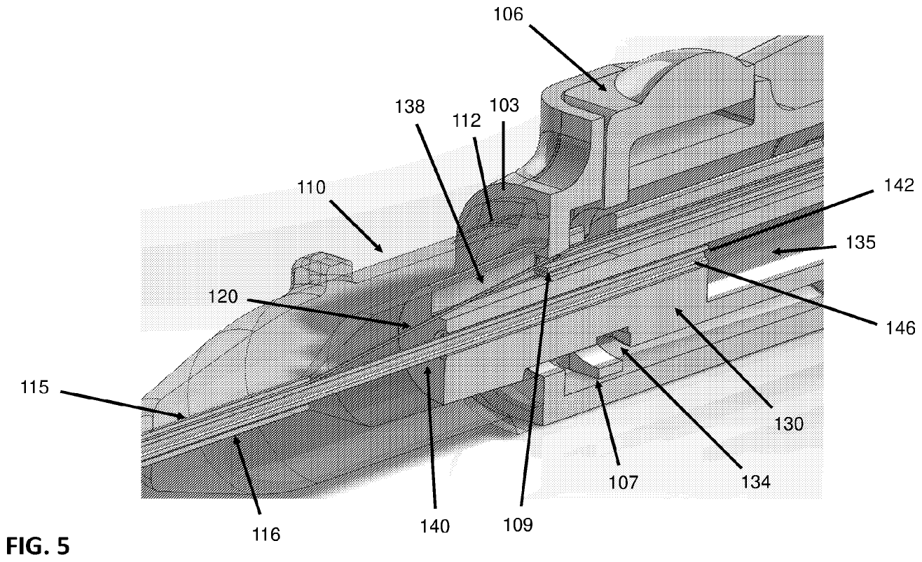

FIG. 5 is a perspective view of a longitudinal cross section of the intravascular access device 100 of FIG. 3 showing additional details of the needle carrier 130 in relation to the actuation button 106 and the guide element 120 to the support rail 109 and the access needle outer wall 148. The catheter hub proximal end 112 is shown coupled to the housing or handle distal end 103. Additional details of the guide element relative to other components may also be appreciated. The guide element 120 extends along the support rail 109, the distal end of the slot 138 into the gap between the needle outer wall and the catheter lumen wall 115. Additionally, the needle 140 is shown in relation to the catheter lumen proximal end 116 and the needle carrier lumen 146. The needle proximal end 142 terminates in the needle carrier 130 into a blood return chamber 135. All or a portion of the needle carrier 130 or the handle 101 may be transparent in order to allow for visual detection of fluid or blood return or bleed back once the needle distal end 144 has entered a target vessel.

FIG. 5 also shows the relationship of the activation latch 107 to the associated needle carrier groove 134. When in the loaded or cocked configuration, the latch 107 would engage with the groove 134 to provide hold back force against the activation element 108. Depression of the activation button 106 will disengage the latch 107 from the needle carrier slot 134 allowing the spring force or stored energy of the activation element 108 to propel the needle carrier 130 towards the handle proximal end 102. This view also shows the termination of the support rail 109 within the housing distal end 103.

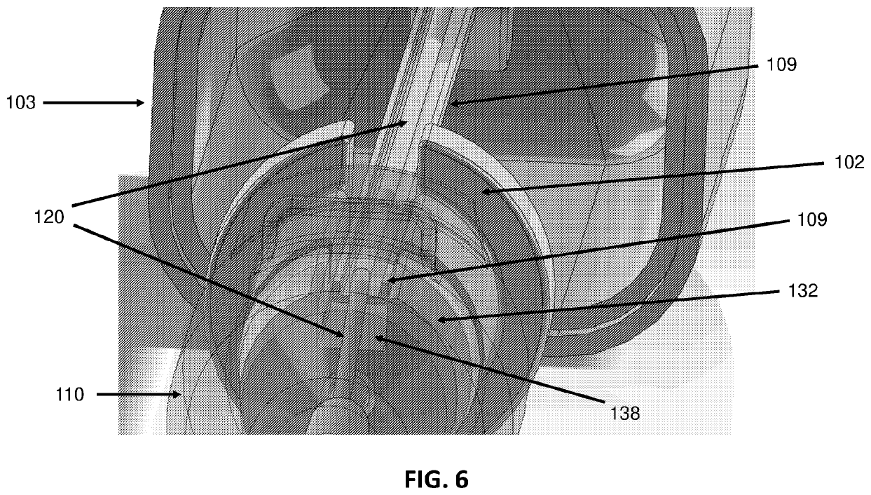

FIG. 6 is an upper view of a portion of the intravascular catheter of FIG. 1 proximal to the view of FIG. 4 showing additional details of the distal end 132 of the needle carrier 130 in relation to the guide element 120 and support rail 109 that are within the needle carrier longitudinal slot 138. The guide element 120 can be seen along the upper surface of the support rail 109, transitioning from the support rail distal end, beyond the needle carrier distal end 132 into the catheter lumen 116 along needle outer wall 148. The arrangement of the support rail 109 to the needle carrier support rail slot 138 allows the support rail to act like a monorail for the needle carrier during retraction (i.e., when the stored energy of the actuation element 108 is released. The support rail 109 also supports the guide element 120 during advancement of the slide 150.

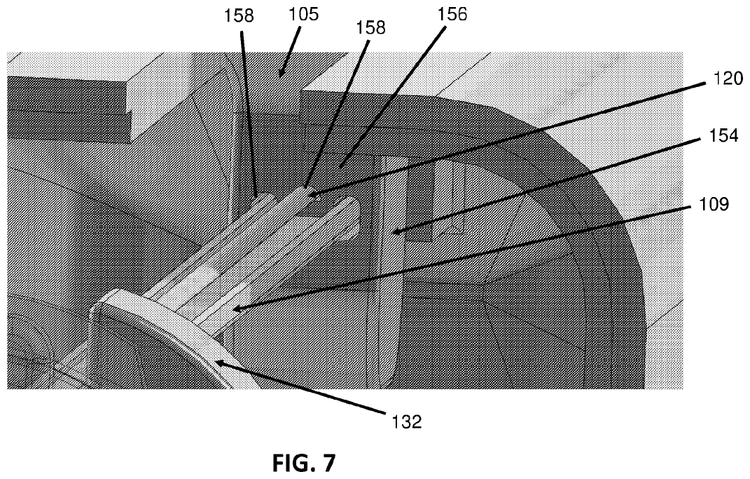

FIG. 7 is an upper view of a portion of an interior view similar to FIG. 6 that shows the proximal end of the needle carrier 132 in relation to the slide 150, guide element 120 and support rail 109. The slider tab 154 of slide 150 extends through slot 105. The slider tab includes a key plate 156 having apertures 158. The apertures 158 are sized, shaped and positioned to accommodate the guide element 120 and features of the support rail 109. Advantageously, the arrangement of the slide 150 and support rail 109 permits the support rail 109 to allow the slide 150 to translate back/forward freely. The guide element 120 has a proximal end 122 that is connected to the slide using the slider tab 154 or other suitable structure. The support rail 109 extends through the slide tab and is connected to the housing proximal end 102. Additionally, this view also shows the relationship of the needle carrier proximal end 132 which is sized and positioned so as to engage with the slide tab 154 during retraction. The rearward movement of the needle carrier will cause the needle carrier proximal end 132 to engage with and move the slide tab 154. The guide element 120 will also move proximally within the housing since the guide element 120 is terminated in the slide 150. The result of this movement (i.e., the proximal movement of the needle carrier 130) simultaneously withdraws the needle and the guide element into the interior of the handle or housing.

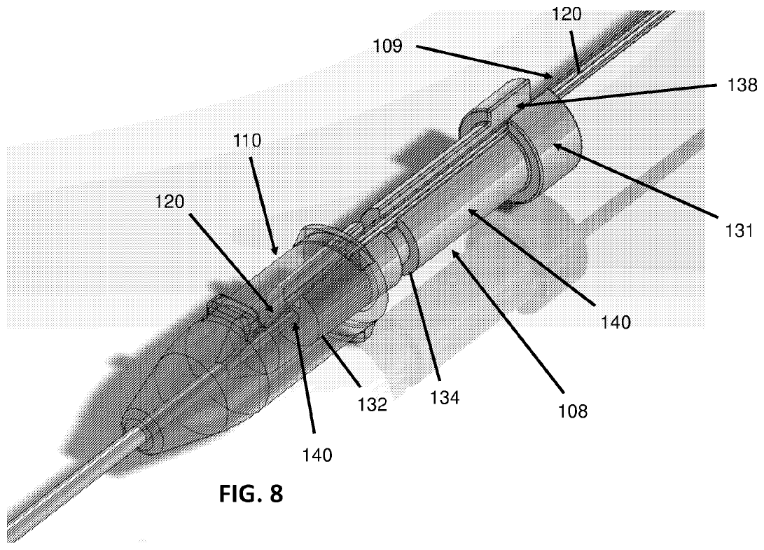

FIG. 8 is an upper perspective view of a distal portion 103 of the intravascular access device 100 of FIGS. 1 and 3 showing the catheter hub 110 interior including the needle carrier 130, the needle 140, the guide element 120 and the support rail 109. This is another view of the placement of the guide element 120 along the support rail 109, beyond the needle carrier and into the catheter lumen. This view also shows the support rail slot 138 along the length of the needle carrier 130 in relation to the needle carrier proximal end 131 and distal groove 134.

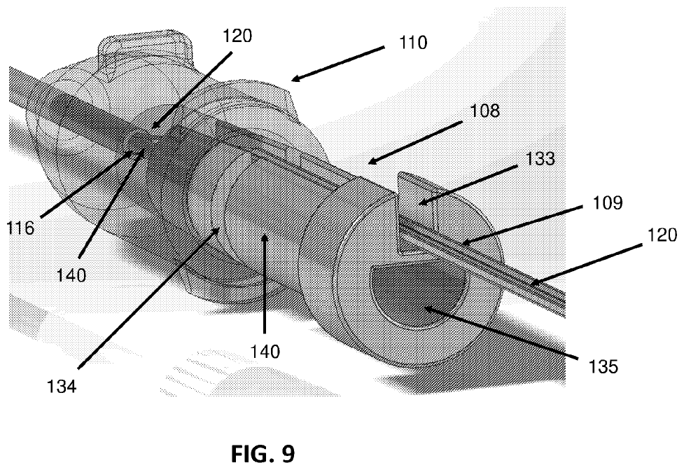

FIG. 9 is perspective proximal view of the needle carrier 130 and other components seen in FIG. 8. The blood chamber 135 is visible within the needle carrier proximal end 131. A normally present cover for the chamber 135 is removed to show the interior.

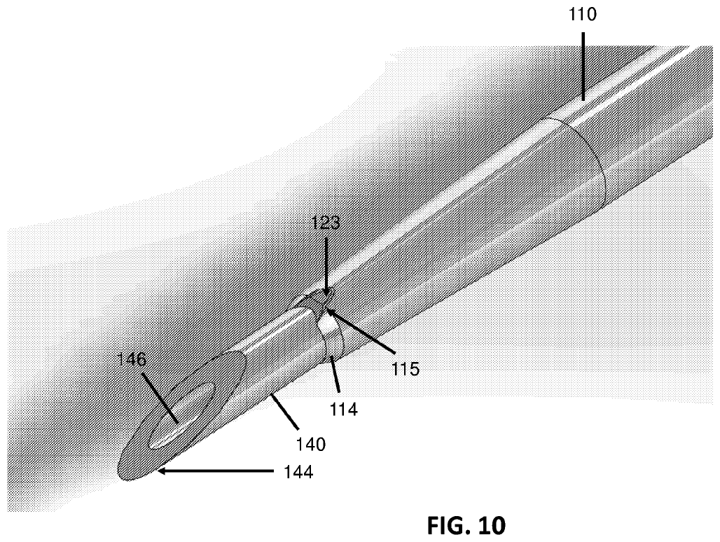

FIG. 10 is an enlarged view of the distal end of the intravascular device 100 of FIG. 1 showing the distal end 144 of the access needle 140 in relation to the distal tip 114 of the catheter with a guide element cut out 115 and the distal end of 123 the guide element 120 within the guide element cut out 115.

The guide element distal end 123 is molded/manufactured to mimic the geometry of catheter tip 114. The guide element 120 location is in the space between catheter lumen inner wall or the inner diameter and needle outer wall or the needle outer diameter. In one embodiment, the guide element 120 is composed of a polymeric material. The guide element 120 may also be pre-shaped or shape set or have a form of shape memory to form a complete loop, a partial loop or multiple loops once extended beyond the needle distal end 144. (See FIGS. 14 and 15).

In a similar way, the catheter distal end includes a guide element cut out 115. The guide element cutout 115 is shaped to accept and conform to the guide element distal end as well as maintain a smooth finish to the distal end of the catheter. The view in FIG. 10 is an example of how the shaped distal end of the guide element conforms to the catheter tip guide element cut out. This is also a view of a uniform transition between the catheter and the contour of the guide element distal end such that the catheter tip to needle transition and the circumference of the catheter tip remain smooth.

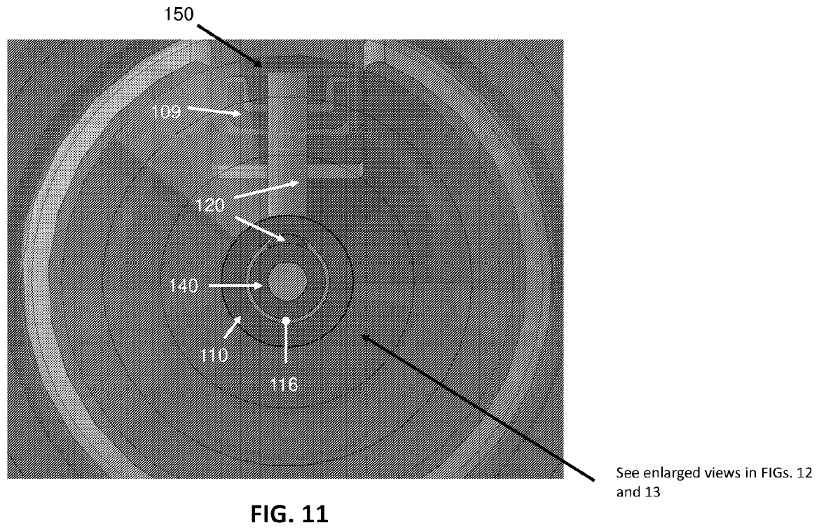

FIG. 11 is a distal end on view of a cross section of the vascular access device of FIG. showing the relationship of the catheter inner wall 115, the guide element 120 and the access needle outer wall 148. The guide element is position within and translates along the space within the catheter lumen along the outer wall of the access needle and the inner wall of the catheter lumen. Optional catheter design includes groove 118 recessed into the catheter lumen inner wall that corresponds to a portion of the guide element upper surface shape 124 or contour. The catheter groove 118 assists with maintaining guide element 120 in position along the catheter and translation along the needle outer wall 148.

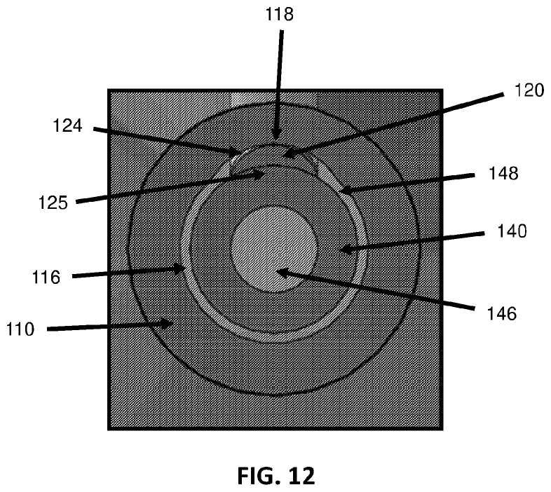

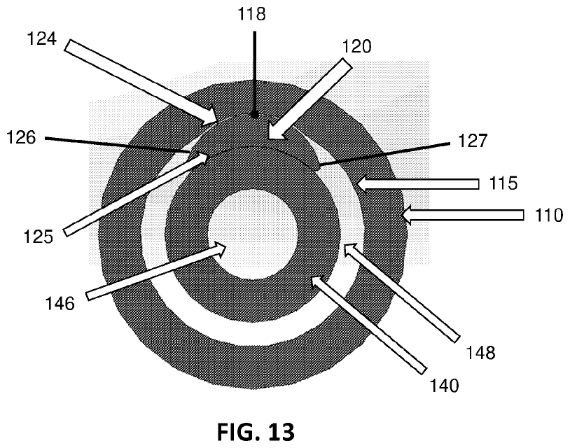

Additionally or optionally, the guiding element design of upper 124 and lower 125 surface contours may correspond to one or both of the catheter lumen interior wall and needle outer wall or to other alignment elements within the catheter lumen. Optionally, in other additional embodiments, the needle outer wall 148 may include a groove or recessed portion for embodiment where a guide element 120 is shaped and configured to operate in an inverted configuration from that shown in this view. See the enlarged views in FIGS. 12 and 13 for additional details of the catheter—guide element—needle alternative embodiments.

FIG. 12 is an enlarged view of the central portion of the cross section view of FIG. 11. This view shows an exemplary location and shape of a recessed portion 118 of the catheter lumen wall 115. This view also shows the guide element 120 in cross section with the upper surface 124 of the guide element adjacent to the lumen wall 115 recessed portion 118 and the guide element lower surface 125 adjacent and conforming to the access needle outer wall 148. The access needle 140 and needle lumen 146 are also shown within the catheter lumen 116.

FIG. 13 is an enlarged view of an alternative configuration the central portion of the cross section view of FIG. 11 but similar to FIG. 12. Those of ordinary skill will appreciate that the guide element may have any of a variety of an upper surface contour 124, a lower surface contour 125, a right side contour 127 and a left side contour 126. Still further, variations in the cross section profile or contours of the guide element 120 may allow for the guide element to extend along and occupy different portions of the space between the catheter inner wall and the needle outer wall. Additionally or optionally, one or more guide element contours may be complementary to an inner wall recess, a guide element track or one or more guide element track features on, in or within a portion of the catheter lumen inner wall or needle outer wall. In one aspect, a guide element upper surface 124 may be contoured or shaped to at least partially conform to catheter lumen inner wall. Additionally or optionally, a guide element lower surface 126 may be shaped to at least partially conform to an access needle exterior wall 148. In the same way, one or more guide element embodiments may employ variations to the right and left contours 126, 127 so as to be adapted to engage with or be complementary to other features provided along the needle or catheter lumen to allow for smooth translation of the guide element along and beyond the access needle distal end 142. In some embodiments, the distance between the guide element left side contour 126 and the guide element right side contour 127 ranges between 0.10 mm to 0.36 mm. This distance is considered the guide element width. Additionally, in still other embodiments, the distance between the guide element upper surface contour 124 and the guide element lower surface contour 125 ranges between 0.127 mm to 0.203 mm This distance is considered the guide element thickness. In some embodiments, the guide element has a length between 100 mm and 200 mm.

FIG. 14 is a perspective view of the distal end of the catheter and access needle of FIG. 10 and FIG. 1 after the slider 50 has been moved from a proximal position as shown in FIG. 1 to a distal position where the guide element 120 is advanced distally along an outer wall 148 of the access needle to form a loop 128 in a position distal to the tip 144 of the access needle 140. In this exemplary embodiment, the loop 128 is formed approximately one inch beyond the distal end 144 of the needle 140. This view also shows how the upper and lower contours of the guide element 120 mate with the catheter guide element cut out 115 and the needle outer wall 148. As such, the guide element 120 may be advanced along an outer surface of an access needle to form a loop 128 beyond the distal end 144 of the needle 140. The deployed guide element 120 may form a partial, complete loop once or more than one complete loop beyond the distal end of the needle. In terms of degree of revolution about the distal end of the needle, a loop 128 may be more than 90 degrees, more than 180 degrees, more than 270 degrees or more than 360 degrees in the case of one full or more than one full revolution. By way of example, the loop 128 in FIGS. 14 and 15 is more than 270 degrees. Still further, exemplary diameters for a loop 128 range from 1 mm to 3 mm.

FIG. 15 is an enlarged view of the guide element loop of FIG. 14. The loop 128 is formed in the distal portion of the guide element 120 when deployed beyond distal most end 144 of access needle 140. This view also shows additional exemplary shapes for the guide element upper surface contour 124, lower surface contour 125, right side contour 127 and the left side contour 126. The upper and lower surface contours 124, 125 of guide element 120 conform to the space between catheter lumen inner wall and outer wall 148 of needle 140.

In some embodiments, the guide element 120 may be formed from PTFE, nylon or another suitable polymer. Additionally or optionally, the guide element 120 may include an additive to enhance visibility under ultrasound guidance. Exemplary additives include, by way of example and not limitation, titanium oxide or barium. Still further, it to be appreciated that a wide array of materials may be used to form the guide element such as a number of polymer blends as well as metals, entirely or partially or of alloys of metals or shape memory materials including metallic and polymer based shape memory materials or of Nitinol or Elgiloy or the like. Still further the dimensions and contours illustrated in the various embodiments of the guide element may also vary along the length of the guide element. For example, the distal most portion of the guide element may have contours and dimensions that are directed to column strength. A mid portion of the guide element 120 may be designed to adapt for the transition from the housing to the catheter lumen without buckling. Still further transitions and variations of contour are possible not only to the guide element portion within the guide catheter but also the interior lumen wall of the catheter may be adapted to aid in the use of the various guide element embodiments. As such, in still further embodiments, the guide element distal tip design forms a uniform transition with the tubular catheter body distal end for maintaining ease of catheter insertion. Additionally, the guide element 120 may have a varying thickness proximal to the catheter hub to assist with column strength. In some embodiments, the distal face of the slide will mate with the proximal face of the needle carrier located inside the housing. In some embodiments, there is a hemostasis valve located inside the tubular catheter proximal hub 110.

As will be appreciated by the various views of the figures, aspects of the present invention provide for improved methods, systems, and assemblies for performing venipuncture, in particular for placing intravascular catheters at a target location in a patient's vein. While the methods, systems, and assemblies will be particularly useful for placement of peripheral venous catheters, such as by placement in a vein on a hand or an arm, they can also be useful with placement of a central venous catheter by insertion into a central vein, such as the internal jugular vein on the neck or the subclavian vein on the chest. Still further, aspects of the vascular access methods, systems, and assemblies may be useful for placement of a catheter in a central or other artery.

More generally, an intravascular catheter assembly in accordance with the principles of the present invention comprises a tubular catheter body, an access needle, a guide element, and a slide for deploying the guide element. The tubular catheter body has a distal end, a proximal end, at least one lumen therebetween and a guide element cutout formed in the distal end. The access needle has a tissue-penetrating distal tip and usually a lumen therethrough. In a first embodiment, the guide element is disposed in the spacing or the lumen between the access needle outer wall and the inner wall of the catheter lumen. In a second embodiment, the guide element is disposed outside and in parallel to the access needle riding along an axial groove, recess or guide structure or structures or guide elements formed in the catheter lumen inner wall. In all embodiments, the guide element has a distal tip configured to (a) conform to the shape and contour of the catheter distal tip in such a manner to form an smooth transition (see FIG. 10) whereby the functionality of the needle catheter combination of providing access is maintained and (b) when extended distally of the catheter along the needle and when distal to the needle will form a partial or complete loop to facilitate advancement of the catheter in the vasculature.

In one embodiment, the slide of the intravascular vascular access assembly is slidably disposed over a support rail containing the guide element. The proximal end of the guide element is attached to the slider. In this way, the guide element may be distally advanced by sliding the slide forwardly or distally along the slot provided in the housing (FIG. 1). This movement moves the distal end of the guide element over the access needle outer surface in order to position the distal tip of the guide element beyond the distal end of the catheter. (FIG. 14). Once in this configuration, the access needle can be fully or partly retracted, or left in place without retraction, and the catheter and the distally projecting guide element can be advanced in tandem to position the distal end of the catheter body at a target site in the venous or other vasculature. By advancing the catheter and the projecting guide element in tandem, the guide element acts as a “fixed” guide element tip, further simplifying the catheter placement protocol. Moreover, the projecting loop of the distal portion of the guide element will aid in preventing or substantially inhibiting the distal tip of the catheter from sticking or kinking on an internal wall of the blood vessel.

In an additional aspect, the intravascular access device includes a housing having at its distal end a needle carrier attached to a proximal end of an access needle as shown in the various views of FIGS. 2, 3, 4, 8 and 9. The access needle 140 is fixedly secured to the needle carrier 130 and the proximal end of the catheter is detachably secured to the distal end of the housing. In this way, the slide 150 can be used to advance the guide element beyond the distal ends of the catheter and the access needle, and the housing, access needle and guide element may be detached and removed from the catheter after the catheter is in place at a desired location in the vasculature.

Coupling the guide element 120 to the slide 150 can be accomplished in any manner which allows advancement or retraction of the slide to impart an equivalent advancement or retraction of the guide element through the space between the catheter lumen and the access needle outer wall. Advantageously, since the guide element is positioned outside of and along the exterior or outer wall of the access needle, the slide can be directly connected to a proximal end of the guide element with minimum interference from the access needle.

In still another aspect of the present invention, a method for introducing an intravascular catheter to a target location in a blood vessel of a patient comprises penetrating a distal tip of an access needle which carries the catheter into the vein. A guide element is within a guide element cutout of the distal tip of the catheter. As a result introduction of the distal most end of the catheter into the blood vessel results in the distal end of the guide element also being positioned into the blood vessel. As such, once bleed back is detected and the needle/catheter combination is advanced such that the distal end of the catheter is within the vessel, the needle may be held stationary. With the needle position held stationary, the slide is advanced from the proximal position shown in FIGS. 1 and 2 into a distal position where the slider is in the distal portion of the slot along the top of the housing. The proximal to distal movement of the slider advances the guide element from the position within the guide cut out in the catheter distal tip (FIG. 10) to the deployed condition with a curved portion, full or partial loop distal to the penetrating tip of the needle as best seen in FIGS. 14 and 15. It is to be appreciated that the guiding element may be pre-shaped to form the loop 128 at a desired or selected distance from the needle distal end 144. In one embodiment, the loop 128 is formed at a minimum distance of about 1 inch or 25.4 mm. Other specific spacing between the needle distal tip and the loop may be provided based on application, clinical need and anatomical considerations of the target lumen.

After the guide element has been advanced, the access needle may optionally be retracted proximally leaving the guide element in place to aid in positioning of the catheter. Optionally, once the catheter is properly located within the blood vessel, the access needle and the guide element are withdrawn completely from the catheter, leaving the catheter in place for a desired medical protocol. In one embodiment, a spring-loaded retraction system or activation element 108 is activated by depressing the actuation button 106. Once the actuation button 106 is depressed or otherwise moved to cause the release of the needle carrier 130, the actuation spring 108 automatically drives the needle carrier 130, needle 140, slide 150 and guide element 120 proximally to withdraw the access needle 140 and guide element 120 from the patient and at least partially or completely into the housing 101. Thereafter the catheter hub 110 is detached from the distal end 103 of the handle or housing 101 leaving the catheter 110 in place for a desired medical protocol as mentioned above. The used housing 101 may then be disposed of using appropriate means.

Additional aspects of the construction and operation of a catheter placement device 100 which includes a housing or handle 101 having mechanism for advancing a guide structure or guide element which carries the catheter where the handle is adapted to automatically retract both the access needle and the guide structure or guide element from the catheter after the placement procedure is complete, a button activated automatic needle and guide withdrawal assembly are described in U.S. Patent Publication US 2008/0300574 and U.S. Pat. No. 9,522,254, each of which is incorporated herein by reference in their entirety.

Still other details of representative intravascular catheter insertion devices and methods are described in U.S. Pat. Nos. 5,704,914 and 5,800,395 and in U.S. Patent Publications US 2010/0094310; US 2010/0210934; and US 2012/0197200, the full disclosure of each of these are incorporated herein by reference in their entirety.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An intravascular catheter assembly, comprising:
A handle having a proximal end and a distal end, a slot in a surface of the handle having a distal end and a proximal end;
An actuation button on the handle coupled to a needle carrier;
a tubular catheter body having a distal end, a guide element cutout in the distal end, a proximal end, and a lumen extending between the proximal end and the distal end, the proximal end of the catheter body coupled to the distal end of the handle;
an access needle disposed in the tubular catheter body lumen having a tissue-penetrating distal tip extending distally beyond the distal end of the tubular catheter body and a proximal end coupled to the needle carrier;
a guide element having a proximal end and a distal end, the guide element positioned within the catheter body lumen and adjacent to an outer wall of the access needle, a distal end portion of the guide element is shaped to provide a uniform transition when the distal end portion is within the guide element cutout, wherein the access needle and the guide element may be withdrawn together from the tubular catheter body after the distal most portion of the guide element has been advanced from the tubular catheter body lumen along an outer wall of the access needle to a position distal to the tissue-penetrating distal tip of the access needle; and
a slide coupled to the proximal end of the guide element so that distal advancement of the slide advances the distal tip end portion of the guide element from a position within the guide element cutout distally along the outer wall of the access needle.

2. The intravascular catheter assembly of claim 1 wherein when the slide is in a distal most position a distal portion of the guide element curves beyond the tissue penetrating distal tip of the access needle.

3. The intravascular catheter assembly of claim 1 wherein when the slide is in a distal most position a distal portion of the guide element curves from a position above the access needle, beyond the tissue penetrating distal tip of the access needle to a position below the access needle.

4. The intravascular catheter assembly of claim 3 wherein the angle of the curve formed by the distal portion of the guide element relative to the tissue penetrating distal tip of the access needle is less than 360 degrees, or wherein the angle of the curve formed by the distal portion of the guide element relative to the tissue penetrating distal tip of the access needle is more than 270 degrees, or wherein the angle of the curve formed by the distal portion of the guide element relative to the tissue penetrating distal tip of the access needle is less than 270 degrees, or wherein the angle of the curve formed by the distal portion of the guide element relative to the tissue penetrating distal tip of the access needle is more than 180 degrees.

5.-7. (canceled)

8. The intravascular catheter assembly according to claim 1, wherein the slide is disposed over a proximal region of the access needle.

9. The intravascular catheter assembly according to claim 1, further comprising a housing attached to a proximal end of the access needle, wherein the slide is disposed over the housing.

10. The intravascular catheter assembly according to claim 9, wherein the access needle is fixedly secured to a distal end of the housing and the tubular catheter body is detachably secured to the distal end of the housing, wherein the slide advances the guide element beyond the distal end of the catheter and wherein the housing, access needle and guide element may be detached and removed from the catheter after the catheter is in place.

11. The intravascular catheter assembly according to claim 10, wherein a proximal region of the catheter is disposed within the housing and wherein the proximal end of the catheter and the proximal end of the access needle are configured to be engaged by the slide to advance the catheter and needle in tandem with the guide element relative to the housing after the guide element has been extended distally beyond the distal end of the catheter.

12. The intravascular catheter assembly according to claim 1, wherein the access needle has a lumen and the guide element is disposed outside of the access needle lumen.

13.-15. (canceled)

16. The intravascular catheter assembly according to claim 1, wherein the guide element is slidably disposed within a guide structure within the catheter body lumen.

17. The intravascular catheter assembly according to claim 16 wherein the guide structure maintains the guide element in a position along an upper most portion of the access needle.

18.-20. (canceled)

21. The intravascular catheter assembly according to claim 16 wherein the guide structure has a distal most portion adjacent to the guide element cutout and a proximal portion wherein the guide structure is continuous along the interior wall of the catheter body lumen from the proximal end to the distal end.

22. The intravascular catheter assembly according to claim 1, wherein the tubular catheter body has a proximal hub with a hemostasis valve, wherein the access needle slidably extends through the hemostasis valve.

23. The intravascular catheter assembly according to claim 22, wherein the slide has a distal face which mates with a proximal face of the needle carrier when the slide is fully advanced distally to extend the guide element.

24. The intravascular catheter assembly according to claim 23, wherein the slide detachably locks to the proximal end of the needle carrier when the distal face mates with the proximal face.

25. The intravascular catheter assembly of claim 1 further comprising:
a first configuration when the slide is in a proximal most position wherein the distal end of the guide element is in the guide element cutout and the tissue penetrating distal tip of the needle is distal to the distal most end of the tubular catheter body;
a second configuration when the slide is in a distal most position wherein the distal most end of the guide element is beyond the guide cutout and curved beyond the tissue penetrating distal tip of the needle; and
a third configuration when the slide is in a position proximal to the distal most position and the needle and the guide element are at least partially retracted into the housing.

26. The intravascular catheter assembly of claim 25 wherein in the first configuration the distal tip of the access needle extends beyond the distal end of the tubular catheter body by a distance in a range from 0.1 mm to 20 mm, and a proximal end of the slide is retracted from the proximal end of the tubular catheter body by a distance in a range from 10 mm to 100 mm.

27. The intravascular catheter assembly of claim 25 wherein in the second configuration the length of the guide element extending beyond the guide cut out of the tubular catheter body is from 5 mm to 100 mm.

28. The intravascular catheter assembly of claim 1 wherein the guide element has a length greater than a width and a width that is greater than a thickness.

29. The intravascular catheter assembly of claim 1 wherein the guide element is made entirely or partially of a polymeric material, a PTFE, a Nylon or wherein the guide element is made entirely or partially of metal, comprising Nitinol, comprising Elgiloy or similar materials.

30. (canceled)

31. The intravascular catheter assembly according to claim 1 the guide element further comprising an upper surface conforming to a curvature of an interior wall of the catheter body lumen and a lower surface conforming to a curvature of an outer wall of the access needle.

32. The intravascular catheter assembly according to claim 1 wherein the catheter is configured for use as a catheter component of an infusion set, a catheter component in a blood collection set, a catheter component in a safety winged blood collection set, or a catheter component in an intravenous catheter.

33.-55. (canceled)
